(19) United States
(12) Patent Application Publication
(10) Pub. No.: US 2005/0245552 A1
(43) Pub. Date: Nov. 3, 2005

(54) N-substituted pyridinone and pyrimidinone derivatives for use as lp-pla2 inhibitors in the treatment of artherosclerosis

(76) Inventors: Colin Andrew Leach, King of Prussia, PA (US); Stephen Allan Smith, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/510,467

(22) PCT Filed: Apr. 10, 2003
(86) PCT No.: PCT/GB03/01544

§ 371 Date: May 25, 2005

(30) Foreign Application Priority Data
Apr. 10, 2002 (GB) 02082790

(51) Int. Cl. A61K 31/513; A61K 31/4415; A61K 31/4439; C07D 43/02; C07D 211/72

(52) U.S. Cl. 514/269; 514/345; 544/309; 546/268.1; 546/296

(57) ABSTRACT

Compounds of formula (I): 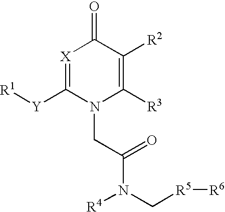
are inhibitors of the enzyme Lp-PLA₂ and are of use in therapy, in particular for treating atherosclerosis. In Formula I R¹, R², R³, R⁴, R⁵, R⁶, X and Y.

The present invention relates to certain novel pyrimidone and pyridone compounds, processes for their preparation, intermediates useful in their preparation, pharmaceutical compositions containing them and their use in therapy, in particular in the treatment of atherosclerosis.

WO 95/00649 (SmithKline Beecham plc) describes the phospholipase A₂ enzyme Lipoprotein Associated Phospholipase A₂ (Lp-PLA₂), the sequence, isolation and purification thereof, isolated nucleic acids encoding the enzyme, and recombinant host cells transformed with DNA encoding the enzyme. Suggested therapeutic uses for inhibitors of the enzyme included atherosclerosis, diabetes, rheumatoid arthritis, stroke, myocardial infarction, reperfusion injury and acute and chronic inflammation. A subsequent publication from the same group further describes this enzyme (Tew D et al, Arterioscler Thromb Vas Biol 1996:16; 591-9) wherein it is referred to as LDL-PLA₂. A later patent application (WO 95/09921, Icos Corporation) and a related publication in Nature (Tjoelker et al, vol 374, 6 Apr. 1995, 549) describe the enzyme PAF-AH which has essentially the same sequence as Lp-PLA₂ and suggest that it may have potential as a therapeutic protein for regulating pathological inflammatory events.

It has been shown that Lp-PLA₂ is responsible for the conversion of phosphatidylcholine to lysophosphatidylcholine, during the conversion of low density lipoprotein (LDL) to its oxidised form. The enzyme is known to hydrolyse the sn-2 ester of the oxidised phosphatidylcholine to give lysophosphatidylcholine and an oxidatively modified fatty acid. Both products of Lp-PLA₂ action are biologically active with lysophosphatidylcholine, in particular having several pro-atherogenic activities ascribed to it including monocyte chemotaxis and induction of endothelial dysfunction, both of which facilitate monocyte-derived macrophage accumulation within the artery wall. Inhibition of the Lp-PLA₂ enzyme would therefore be expected to stop the build up of these macrophage enriched lesions (by inhibition of the formation of lysophosphatidylcholine and oxidised free fatty acids) and so be useful in the treatment of atherosclerosis.

A recently published study (WOSCOPS—Packard et al, N. Engl. J. Med. 343 (2000) 1148-1155) has shown that the level of the enzyme Lp-PLA₂ is an independent risk factor in coronary artery disease.

The increased lysophosphatidylcholine content of oxidatively modified LDL is also thought to be responsible for the endothelial dysfunction observed in patients with atherosclerosis. Inhibitors of Lp-PLA₂ could therefore prove beneficial in the treatment of this phenomenon. An Lp-PLA₂ inhibitor could also find utility in other disease states that exhibit endothelial dysfunction including diabetes, hypertension, angina pectoris and after ischaemia and reperfusion.

In addition, Lp-PLA₂ inhibitors may also have a general application in any disorder that involves activated monocytes, macrophages or lymphocytes, as all of these cell types express Lp-PLA₂. Examples of such disorders include psoriasis.

Furthermore, Lp-PLA₂ inhibitors may also have a general application in any disorder that involves lipid oxidation in conjunction with Lp-PLA₂ activity to produce the two injurious products, lysophosphatidylcholine and oxidatively modified fatty acids. Such conditions include the aforementioned conditions atherosclerosis, diabetes, rheumatoid arthritis, stroke, myocardial infarction, ischaemia, reperfusion injury and acute and chronic inflammation.

Patent applications WO 96/12963, WO 96/13484, WO 96/19451, WO 97/02242, WO 97/217675, WO 97/217676, WO 96/41098, and WO 97/41099 (SmithKline Beecham plc) disclose inter alia various series of 4-thionyl/sulfinyl/sulfonyl azetidinone compounds which are inhibitors of the enzyme Lp-PLA₂. These are irreversible, acylating inhibitors (Tew et al, Biochemistry, 37, 10087, 1998).

A further class of compounds has now been identified which are non-acylating inhibitors of the enzyme Lp-PLA₂. Thus, WO 99/24420, WO 00/10980, WO 00/66566, WO 00/66567, WO 00/68208 and PCT/EP01/11562 (unpublished at the priority date of the present application) (SmithKline Beecham plc) disclose classes of pyrimidone compounds. PCT/EP01/11610 (SmithKline Beecham plc), also unpublished at the priority date of the instant application, discloses a class of pyridone compounds. We have now found that the amide nitrogen substituent on both the pyrimidone and pyridone ring scaffolds may be replaced, to give compounds having good activity as inhibitors of the enzyme Lp-PLA₂.

Accordingly, the present invention provides a compound of formula (I): 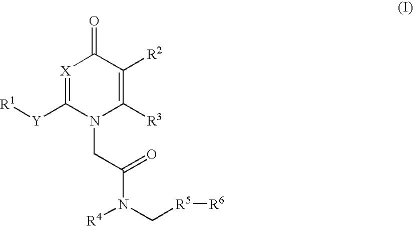
in which:
R¹ is an aryl group, optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR⁷COR⁸, CONR⁹R¹⁰, SO₂NR⁹R¹′, NR⁷SO₂R⁸, NR⁹R¹⁰, mono to perfluoro-C₍₁₋₄₎alkyl, mono to perfluoro-C₍₁₋₄₎alkoxyaryl, and arylC₍₁₋₄₎alkyl;
R² is halogen, C₍₁₋₃₎alkyl, C₍₁₋₃₎alkoxy, hydroxyC₍₁₋₃₎alkyl, C₍₁₋₃₎alkylthio, C₍₁₋₃₎alkylsulphinyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, C₍₁₋₃₎alkylcarboxy, C₍₁₋₃₎alkylcarboxyC₍₁₋₃₎alkyl, and
R³ is hydrogen, halogen, C₍₁₋₃₎alkyl, or hydroxyC₍₁₋₃₎alkyl; or
R² and R³ together with the pyridone or pyrimidone ring carbon atoms to which they are attached form a fused 5- or 6-membered carbocyclic ring; or
R² and R³ together with the pyridone or pyrimidone ring carbon atoms to which they are attached form a fused benzo or heteroaryl ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy or C₍₁₋₄₎alkylthio, or mono to perfluoro-C₍₁₋₄₎alkyl;
R⁴ is Het-C₍₀₋₄₎alkyl in which Het is a 5- to 7-membered saturated heterocyclyl ring comprising N and optionally O or S, and in which N is substituted by C₃₋₈cycloalkyl or C₍₁₋₆₎alkyl further substituted by 1, 2 or 3 substituents selected from R¹¹, COOR¹¹, COOCH₂R¹¹, COR¹¹, CN, CONR¹²R¹³, C₃₋₈cycloalkyl, vinyl optionally substituted by halogen or C₍₁₋₃₎alkyl and a 5- to 7-membered saturated heterocyclyl ring comprising N in which N may be substituted by C₁₋₃alkyl;
R⁵ is an aryl or a heteroaryl ring optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR COR⁸, CONR⁹R⁶⁰, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy;
R⁶ is an aryl or a heteroaryl ring which is further optionally substituted by 1, 2, 3 or 4 substituents which may be the same or different selected from C₍₁₋₆₎alkyl, C₍₁₋₆₎alkoxy, C₍₁₋₆₎alkylthio, C₍₁₋₆₎alkylsulfonyl, arylC₍₁₋₆₎alkoxy, hydroxy, halogen, CN, COR⁷, carboxy, COOR⁷, CONR⁹R¹⁰, NR⁷COR⁸, SO₂NR⁹R¹⁰, NR SO₂R⁸, NR⁹R¹⁰, mono to perfluoro-C₍₁₋₄₎alkyl and mono to perfluoro-C₍₁₋₄₎alkoxy, or C₍₅₋₁₀₎alkyl;
R⁷ and R⁸ are independently hydrogen or C₍₁₋₁₂₎alkyl, for instance C₍₁₋₄₎alkyl (e.g. methyl or ethyl);
R⁹ and R¹⁰ which may be the same or different is each selected from hydrogen, or C₍₁₋₂₎alkyl, or R⁹ and R¹⁰ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl, e.g benzyl, for instance morpholine or piperazine;
R¹¹ is an unsubstituted 5- or 6-membered heteroaryl or an unsubstituted 6-membered aryl, or a 5- or 6-membered heteroaryl or a 6-membered aryl substituted by one or more R¹⁴.

R¹² is selected from hydrogen or C₁₋₃alkyl;
R¹³ is selected from phenyl optionally substituted by halogen, C₁₋₆alkyl, C₁₋₆alkoxy or cyano, or C₅₋₇cycloalkyl;
R¹⁴ is selected from the group consisting of halogen, CF₃, C₁₋₆alkyl, C₁₋₆alkoxy or cyano;
X is CH or nitrogen; and
Y is a C₍₂₋₄₎alkylene group (optionally substituted by 1, 2 or 3 substituents selected from methyl and ethyl), CH═CH, or (CH₂)ₙS where n is 1, 2 or 3.

In another aspect the invention provides a compound of formula (1) as defined above in which R¹ is an aryl group, optionally substituted by 1, 2, 3 or 4 substituents which may be the same or different selected from halogen, C₍₁₋₆₎alkyl, trifluoromethyl or C₍₁₋₆₎alkoxy.

Representative examples of R¹ when an aryl group include phenyl. Preferably, R¹ is phenyl optionally substituted by 1, 2, 3 or 4 halogen substituents, preferably, from 1 to 3 fluoro, more preferably, 2,3-difluoro or 4-fluoro.

In another aspect the present invention provides a compound of formula (1) as defined above in which, when X is CH, R² and R³ together with the pyridone ring carbon atoms to which they are attached form a fused benzo or pyrido ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy or C₍₁₋₄₎alkylthio, or mono to perfluoro-C₍₁₋₄₎alkyl.

Representative examples of R² and R³ include when R² and R³, together with the pyridone ring carbon atoms to which they are attached, form an unsubstituted fused benzo or pyrido ring.

In another aspect the present invention provides a compound of formula (I) as defined above in which, when X is nitrogen, R² and R³ together with the pyrimidone ring carbon atoms to which they are attached form a fused 5-membered carbocyclic (cyclopentenyl) or benzo ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy or C₍₁₋₄₎alkylthio, or mono to perfluoro-C₍₁₋₄₎alkyl.

Representative examples of R² and R³ include when R² and R³, together with the pyrimidone ring carbon atoms to which they are attached, form an unsubstituted fused benzo or cyclopentenyl ring.

In another aspect the present invention provides a compound of formula (I) as defined above in which R⁴ is Het-C₍₀₎alkyl in which Het is a six-membered saturated heterocyclyl ring comprising nitrogen in which the nitrogen is substituted by C₃₋₈cycloalkyl or C₍₁₋₂₎alkyl substituted by a single substituent selected from R¹¹, COOR¹¹, COOCH₂R¹¹, COR¹¹, CN, CONR¹²R¹³, C₃₋₈cycloalkyl, vinyl optionally substituted by halogen or methyl and a 5- or 6-membered saturated heterocyclyl ring comprising N in which the nitrogen may be substituted by methyl.

Representative examples of R⁴ include piperidin-4-yl substituted at the 1-position by methyl which is further substituted by pyridyl, thiazol-2-yl, cyano, 2-methylthiazol-4-yl, 2-chlorothiazol-4-yl, 1-methylpiperidin-3-yl, cyclopropyl, phenyl, 5-methyl-isoxazol-3-yl, 1-chlorovinyl, 2,2-dimethylvinyl, COOR¹¹, COR¹¹ and CONR¹²R¹³.

Representative examples of R⁴ include piperidin-4-yl substituted at the 1-position by ethyl which is further substituted by 1-methyl-pyrrolidin-2-yl, pyrazol-1-yl, imidazol-1-yl and vinyl.

In another aspect the present invention provides a compound of formula (I) as defined above in which R⁵ is phenyl or pyridyl.

Representative examples of R⁵ include phenyl.

In another aspect the present invention provides a compound of formula (I) as defined above in which R⁶ is phenyl substituted by mono to perfluoro-C₍₁₋₄₎alkyl, halogen or C₍₁₋₆₎alkyl.

Representative examples of R⁶ include phenyl substituted by trifluoromethyl at the 4-position.

Preferably, R⁵ and R⁶ together form a 4-(phenyl)phenyl or a 2-(phenyl)pyridinyl substituent in which the remote phenyl ring may be optionally substituted by trifluoromethyl, preferably at the 4-position.

Representative examples of R¹¹ include phenyl, pyridyl, thiazolyl, pyrazolyl, imidazolyl and isoxazolyl.

Representative examples of R¹² include hydrogen and methyl.

Representative examples of R¹³ include cyclohexyl and phenyl.

Representative examples of R¹⁴ include methyl, chloro, methoxy and cyano.

In another aspect, the present invention provides a compound of formula (I) as defined above in which Y is a C₍₂₋₄₎alkylene group or CH₂S.

Representative examples of Y when X is CH or nitrogen include CH₂S and (CH₂)₂.

It is to be understood that the invention covers all combinations of particular aspects of the invention as described hereinabove.

It will be appreciated that compounds of the present invention may comprise one or more chiral centres so that stereoisomers may be formed. The present invention encompasses all stereoisomers of the compounds of formula (I) including geometric isomers and optical isomers (eg. diastereoisomers and enantiomers) whether as individual stereoisomers isolated such as to be substantially free of the other stereoisomers (ie. pure) or as mixtures thereof including racemic modifications. An individual stereoisomer isolated such as to be substantially free of other stereoisomer (ie. pure) will preferably be isolated such that less than 10% preferably less than 1% especially less than 0.1% of the other stereoisomers is present.

Certain compounds of formula (1) may exist in one of several tautomeric forms. It will be understood that the present invention encompasses all tautomers of the compounds of formula (I) whether as individual tautomers or as mixtures thereof.

It will be appreciated that in some instances, compounds of the present invention may include a basic function such as an amino group as a substituent. Such basic functions may be used to form acid addition salts, in particular pharmaceutically acceptable salts. Pharmaceutically acceptable salts include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, taurocholic acid, benzenesulfonic, p-toluenesulfonic, hydrochloric, hydrobromic, sulfuric, cyclohexylsulfamic, phosphoric and nitric acids.

It will be appreciated that in some instances, compounds of the present invention may include a carboxy group as a substituent. Such carboxy groups may be used to form salts, in particular pharmaceutically acceptable salts. Pharmaceutically acceptable salts include those described by Berge, Bighley, and Monkhouse, J. Pharm. Sci., 1977, 66, 1-19. Preferred salts include alkali metal salts such as the sodium and potassium salts.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the term “aryl” refers to, unless otherwise defined, a mono- or bicyclic aromatic ring system containing up to 10 carbon atoms in the ring system, for instance phenyl or naphthyl.

When used herein, the term “heteroaryl” refers to a mono- or bicyclic heteroaromatic ring system comprising up to four, preferably 1 or 2, heteroatoms each selected from oxygen, nitrogen and sulphur. Each ring may have from 4 to 7, preferably 5 or 6, ring atoms. A bicyclic heteroaromatic ring system may include a carbocyclic ring.

When used herein, the terms “halogen” and “halo” include fluorine, chlorine, bromine and iodine and fluoro, chloro, bromo and iodo, respectively.

When used herein the term “5-membered heteroaryl” means a heteroaryl selected from the following:

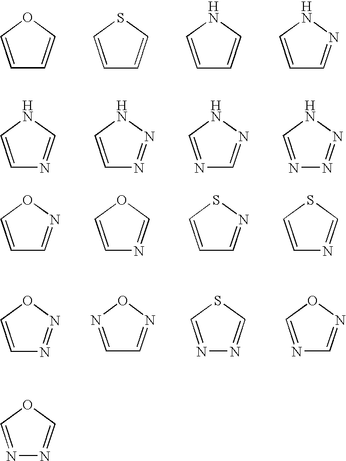

The term “6-membered heteroaryl” means a heteroaryl selected from the following:

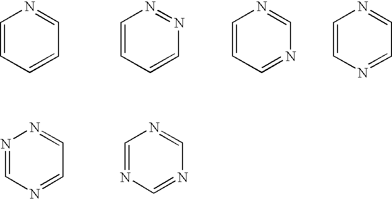

The term “6-membered aryl” means:

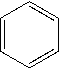

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are re-crystallised from organic solvents, solvent of crystallisation may be present in the crystalline product. This invention includes within its scope such solvates. Similarly, some of the compounds of this invention may be crystallised or re-crystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (1).

Compounds of the present invention are inhibitors of the enzyme lipoprotein associated phospholipase A₂ (Lp-PLA₂) and as such are expected to be of use in therapy, in particular in the treatment of atherosclerosis. In a further aspect therefore the present invention provides a compound of formula (1) for use in therapy.

The compounds of formula (I) are inhibitors of lysophosphatidylcholine production by Lp-PLA₂ and may therefore also have a general application in any disorder that involves endothelial dysfunction, for example atherosclerosis, diabetes, hypertension, angina pectoris and after ischaemia and reperfusion. In addition, compounds of formula (1) may have a general application in any disorder that involves lipid oxidation in conjunction with enzyme activity, for example in addition to conditions such as atherosclerosis and diabetes, other conditions such as rheumatoid arthritis, stroke, inflammatory conditions of the brain such as Alzheimer's Disease, myocardial infarction, ischaemia, reperfusion injury, sepsis, and acute and chronic inflammation.

Further applications include any disorder that involves activated monocytes, macrophages or lymphocytes, as all of these cell types express Lp-PLA₂. Examples of such disorders include psoriasis.

Accordingly, in a further aspect, the present invention provides for a method of treating a disease state associated with activity of the enzyme Lp-PLA₂ which method involves treating a patient in need thereof with a therapeutically effective amount of an inhibitor of the enzyme. The disease state may be associated with the increased involvement of monocytes, macrophages or lymphocytes; with the formation of lysophosphatidylcholine and oxidised free fatty acids; with lipid oxidation in conjunction with Lp-PLA₂ activity; or with endothelial dysfunction.

Compounds of the present invention may also be of use in treating the above mentioned disease states in combination with an anti-hyperlipidaemic, anti-atherosclerotic, anti-diabetic, anti-anginal, anti-inflammatory, or anti-hypertension agent or an agent for lowering Lp(a). Examples of the above include cholesterol synthesis inhibitors such as statins, anti-oxidants such as probucol, insulin sensitisers, calcium channel antagonists, and anti-inflammatory drugs such as NSAIDs. Examples of agents for lowering Lp(a) include the aminophosphonates described in WO 97/02037, WO 98/28310, WO 98/28311 and WO 98/28312 (Symphar SA and SmithKline Beecham).

A preferred combination therapy will be the use of a compound of the present invention and a statin. The statins are a well known class of cholesterol lowering agents and include atorvastatin, simvastatin, pravastatin, cerivastatin, fluvastatin, lovastatin and rosuvastatin (also referred to as S-4522 or ZD 4522, Astra Zeneca). The two agents may be administered at substantially the same time or at different times, according to the discretion of the physician.

A further preferred combination therapy will be the use of a compound of the present invention and an anti-diabetic agent or an insulin sensitiser, as coronary heart disease is a major cause of death for diabetics. Within this class, preferred compounds for use with a compound of the present invention include the PPARgamma activators, for instance G1262570 (GlaxoSmithKline) and the glitazone class of compounds such as rosiglitazone (Avandia, GlaxoSmithKline), troglitazone and pioglitazone.

In therapeutic use, the compounds of the present invention are usually administered in a standard pharmaceutical composition. The present invention therefore provides, in a further aspect, a pharmaceutical composition comprising a compound of formula (I) and a pharmaceutically acceptable carrier, optionally with one or more other therapeutic compounds such as a statin or an anti-diabetic.

Suitable pharmaceutical compositions include those which are adapted for oral or parenteral administration or as a suppository. Compounds of formula (I) which are active when given orally can be formulated as liquids, for example syrups, suspensions or emulsions, tablets, capsules and lozenges. A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavouring or colouring agent. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule. Typical parenteral compositions consist of a solution or suspension of the compound of formula (I) in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of formula (I) which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule. Each dosage unit for oral administration contains preferably from 1 to 500 mg (and for parenteral administration contains preferably from 0.1 to 25 mg) of a compound of the formula (1). The daily dosage regimen for an adult patient may be, for example, an oral dose of between 1 mg and 1000 mg, preferably between 1 mg and 500 mg, or an intravenous, subcutaneous, or intramuscular dose of between 0.1 mg and 100 mg, preferably between 0.1 mg and 25 mg, of the compound of the formula (I), the compound being administered 1 to 4 times per day. Suitably the compounds will be administered for a period of continuous therapy, for example for a week or more.

A compound of formula (I) may be prepared by reacting an acid compound of formula (II): 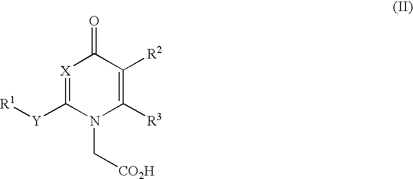
in which X, Y, R¹, R² and R³ are as hereinbefore defined,
with an amine compound of formula (III):
R⁶—R⁵—CH₂NHR⁴ (III)
in which R⁴, R⁵ and R⁶ are as hereinbefore defined; under amide forming conditions.

Suitable amide forming conditions are well known in the art and include treating the acid of formula (1H) with the amine of formula (III) in the presence of a coupling agent such as 1-(3-dimethyl-aminopropyl)-3-ethylcarbodimide (DEC) and 1-hydroxybenzotriazole (HOBt), or O-(7-azabenzotriazol-1-yl)-N,N,N′,N′-tetramethyluronium hexafluorophosphate (HATU) and di-isopropylethylamine, in an aprotic solvent such as dichloromethane or dimethylformamide.

It will be appreciated by those skilled in the art that amines of formula (III) are either known compounds or may be prepared by literature methods such as reductive amination between suitable carbonyl and amine precursors, employing an appropriate reducing agent such as sodium triacetoxyborohydride or sodium borohydride. Such methods are described in “Comprehensive Organic Transformations: a guide to functional group preparations” by Richard Larock (VCH, 1989), incorporated herein by reference.

It will be appreciated by those skilled in the art that compounds of formula (I) may be prepared by interconversion, utilising suitable precursors of compounds of formula (I). Specifically, compounds of formula (I) wherein R⁴ is Het-C₍₀₋₄₎alkyl in which Het is a 5- to 7-membered saturated heterocyclyl ring comprising N and optionally O or S, and in which N may be substituted by C₍₁₋₆₎alkyl which may be further substituted, may be synthesised from precursor compounds wherein R⁴ is Het-C₍₀₋₄₎alkyl in which Het is a 5- to 7-membered saturated heterocyclyl ring comprising N and optionally O or S, and in which N is unsubstituted, by alkylation. Alkylations are well known to those skilled in the art and are described in many standard organic texts, such as ‘Advanced Organic Chemistry’ by Jerry March, fourth edition (Wiley, 1992), incorporated herein by reference. Thus, a process for preparing a compound of formula (I) by interconversion of another compound of formula (1) constitutes a further aspect of the present invention.

A compound of formula (1) may be readily prepared from a corresponding ester of formula (IV): 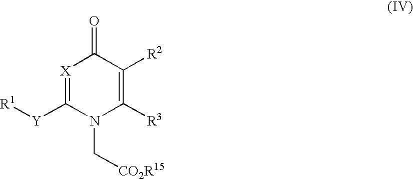
in which X, Y, R¹, R² and R³ are as hereinbefore defined, and R¹⁵ is benzyl or C₍₁₋₆₎alkyl, for example ethyl or t-butyl, by treating with a de-esterifying agent, for instance, when R¹⁵ is t-butyl, trifluoroacetic acid or when R¹⁵ is ethyl or benzyl, sodium hydroxide in dioxan.

The overall synthesis of compounds of formula (I) is illustrated in the following scheme wherein R¹ to R¹⁵ are as hereinbefore defined:

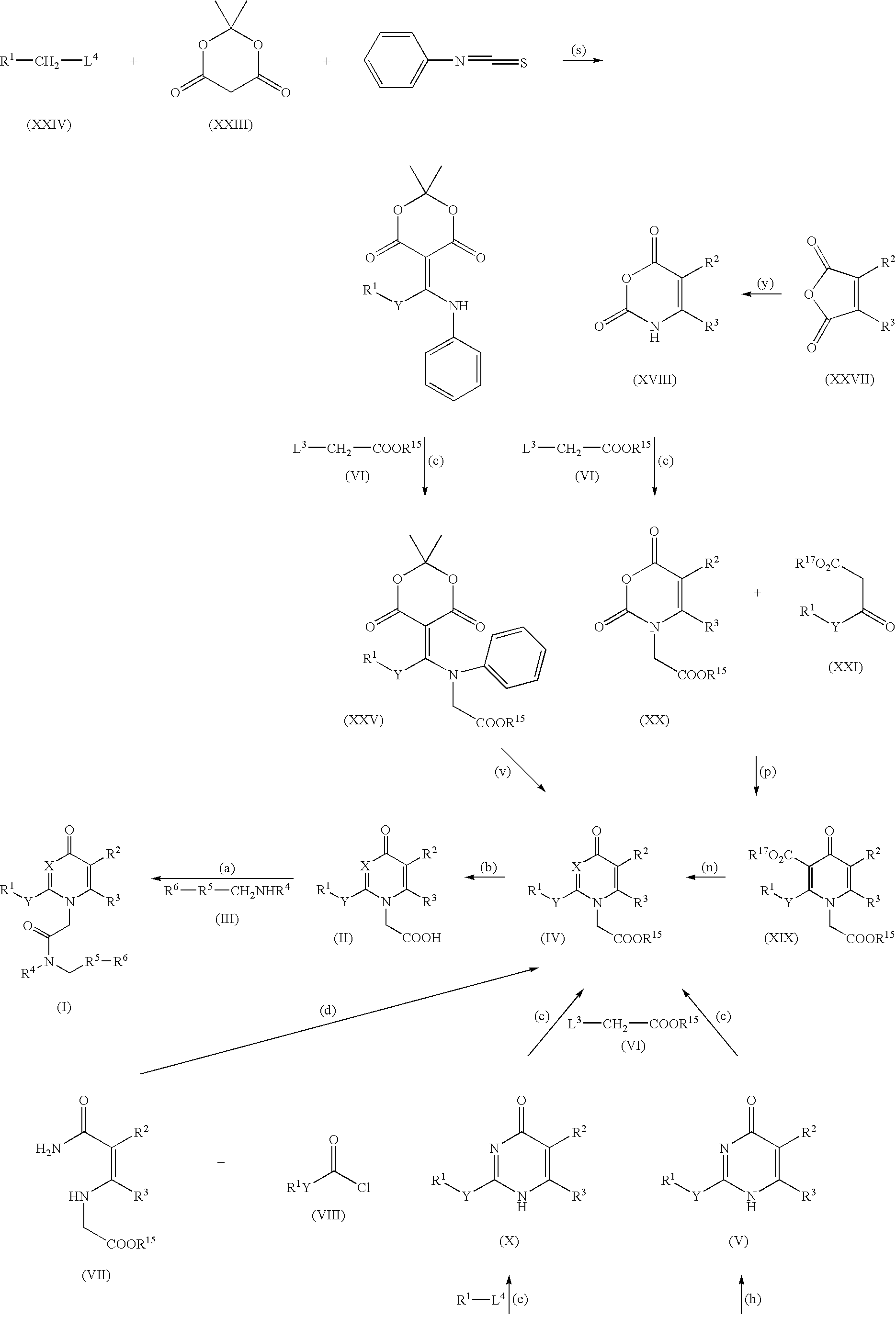

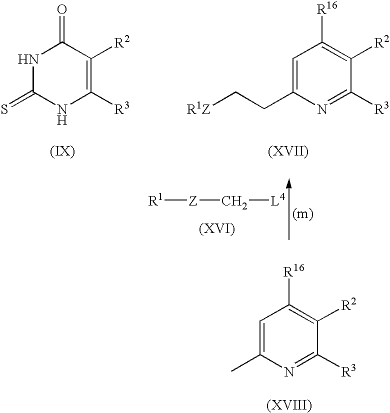

Referring to the scheme when X is CH, the ester (IV) is usually prepared by N-1 alkylation of (V) using (VI), in which L³ is a leaving group (e.g. Br) and R¹⁵ is as hereinbefore defined e.g. (VI) is t-butyl bromoacetate or ethyl bromoacetate, in the presence of a base e.g. BuLi in THF, sodium hydride in N-methyl pyrrolidinone (NMP), or a secondary or teriary amine such as di-isopropylethylamine, in an inert solvent such as dichloromethane (step c).

Alternatively, when X is CH, Y is CH₂S, and R² and R³, together with the pyridone ring carbon atoms to which they are attached, form a fused benzo ring, intermediate (IV) may be synthesised from known starting materials by steps (s), (c) and (v) in which:
(s) treatment of Meldrum's acid (XXIII) with sodium hydride at low temperature, followed by reaction with phenylisothiocyanate and subsequent treatment with R¹CH₂-L⁴, where L⁴ is a leaving group;
(c) as hereinbefore discussed;
(v) treatment of (XXV) with trifluoroacetic acid.

When X is CH and Y is alkylene, it is preferable to use steps (m), (h) and (c) (intermediates (XVIII), (XVII) and (V)) or steps (n) and (p) (intermediates (XIX), (XX), (XXI)) in which:
(m) chain extension of a 2-alkyl pyridine, e.g. where Y=ZCH₂CH₂ by treatment of a 2-methylpyridine (XVIII) with R¹-Z-CH₂-L⁴ (XVI) in which L⁴ is a leaving group and a strong base, such as BuLi, in THF.
(h) transformation of a 4-substituted pyridine into a 4-pyridone e.g. by treatment of (XVII) R¹⁶=Cl with aq HCl and dioxan, or deprotection of R¹⁶=Oallyl, e.g. using (Ph₃P)₃RhCl when in aq. ethanol.
(c) as hereinbefore described.

In the alternative route, the 3-ester group is removed from intermediate (XIX) R¹⁷=C₍₁₋₆₎alkyl by heating in diphenyl ether where R¹⁷=tBu (step n); Intermediate (XIX) is formed from the 2,6-dioxo-1,3-oxazine (XX) and ester (XXI) by treatment with a base such as NaH in DMF or 1,8-diazabicyclo[5.4.0]undec-7-ene in dichloromethane (step p).

Synthesis of (XX) from known starting materials may be achieved via steps (y) and (c) in which:
(y) treatment of (XXVII) with azidotrimethylsilane in tetrahydrofuran or dichloromethane;
(c) as hereinbefore described.

When X is nitrogen and Y is CH₂S it is preferable to use steps (e) and (c) (intermediates (DC), (X)) in which:
(e) thioether forming reaction. Treatment of (IX) with R¹-L⁴ in the presence of a base such as sodium ethoxide or potassium carbonate, preferably in a solvent such as ethanol, dimethyl formamide, or acetone, or a secondary or tertiary amine base such as di-isopropylethylamine, in a solvent such as dichloromethane.
(c) as hereinbefore described.

When X is nitrogen and Y is (CH₂)₂ it is preferable to react intermediate (VII) with intermediate (VIII) (step (d)) under standard pyrimidone ring forming conditions, in a solvent such as benzene.

It will be appreciated by those skilled in the art that all other starting materials and intermediates are either known compounds or may be prepared by literature methods, such as those described in “Comprehensive Organic Transformations: a guide to functional group preparations” by Richard Larock (VCH, 1989), incorporated herein by reference.

As will be appreciated by those skilled in the art it may be necessary or desirable at any stage in the synthesis of compounds of formula (I) to protect one or more sensitive groups in the molecule so as to prevent undesirable side reactions. Thus, the above processes may require deprotection as an intermediate or final step to yield the desired compound. Thus, according to another process, a compound of formula (I) may be prepared by subjecting a protected derivative of a compound of formula (I) to reaction to remove the protecting group or groups present, constituting a further aspect of the present invention.

The protecting groups used in the preparation of compounds of formula (1) may be used in conventional manner. See for example, those described in ‘Protective Groups in Organic Synthesis’ by Theodora W Green and Peter G M Wuts, second edition, (John Wiley and Sons, 1991), incorporated herein by reference, which also describes methods for the removal of such groups. Thus, hydroxyl groups may be protected using any conventional hydroxyl protecting group. Examples of suitable hydroxyl protecting groups includes groups selected from alkyl (e.g. t-butyl or methoxymethyl), aralkyl (e.g. benzyl, diphenylmethyl or triphenylmethyl), heterocyclic groups such as tetrahydropyranyl, acyl (e.g. acetyl or benzoyl) and silyl groups such as trialkylsilyl (e.g. t-butyldimethylsilyl). The hydroxyl protecting groups may be removed by conventional techniques. Thus, for example alkyl, silyl, acyl and heterocyclic groups may be removed by solvolysis, e.g. by hydrolysis under acidic or basic conditions. Aralkyl groups such as triphenylmethyl may be similarly be removed by solvolysis, e.g. by hydrolysis under acidic conditions. Aralkyl groups such as benzyl may be cleaved by hydrogenolysis in the presence of a Noble metal catalyst such as palladium-on-charcoal.

The present invention will now be illustrated by the following examples.

### EXAMPLES

The structure and purity of the intermediates and examples was confirmed by ¹H-NMR and (in nearly all cases) mass spectroscopy, even where not explicitly indicated below

### Intermediate A1 4-(4-Trifluoromethylphenyl)benzaldehyde

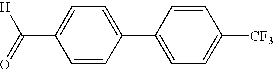

A 3 L 3-neck flask fitted with top stirrer, condenser and argon inlet/outlet was charged with 4-trifluoromethybenzene boronic acid (90.0 g, 0.474 mol), 4-bromobenzaldehyde (83.29 g, 0.450 mol) and 1,2-dimethoxyethane (1.3 L), followed by 2M aqueous sodium carbonate (474 mL) and palladium acetate (5.32 g, 0.0237 mol). The stirring mixture was heated to reflux for 4 h under argon, then allowed to cool to room temperature over 16 h. The reaction mixture was filtered through hyflo. The filtrate was diluted with saturated brine and extracted 3× with ethyl acetate. The combined extracts were dried over magnesium sulfate and filtered through hyflo, giving a clear orange filtrate which was evaporated to a solid (ca. 120 g, crude). Flash chromatography (silica, 10-50% dichloromethane in pet. ether, 10% steps) gave a white solid which dissolved in hexane (500 mL) on boiling. Crystallisation, finally in ice, gave the title compound as a solid which was filtered off, washed with ice cold hexane and dried, (86.33 g, 77%). ¹H-NMR (CDCl₃) δ 7.77-8.03 (8H, m), 10.09 (1H, s).

### Intermediate A2—4-(4-Trifluoromethylphenyl)benzonitrile

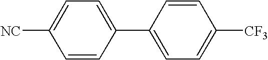

Prepared by the method of intermediate A1 using 4-trifluoromethylbenzeneboronic acid and 4-bromobenzonitrile. ¹H-NMR (DMSO) δ 7.99-7.94 (6H, m) 7.86 (2H, d); MS(APCI+) found (M+1)=248, C₁₄H₈NF₃ requires 247.

### Intermediate A3—4-(4-Trifluoromethylphenyl)benzylamine hydrochloride salt

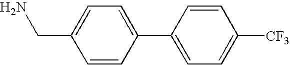

To a solution of intermediate A2 (96.7 g, 0.39 mol) in absolute ethanol (51) and concentrated hydrochloric acid (200 ml) was added 10% palladium on charcoal (30.0 g, 54% H₂O paste). The mixture was stirred under 50 psi hydrogen for 16 h. Additional 10% palladium on charcoal (25.0 g, 54% H₂O paste) was added and the mixture was stirred under 50 psi hydrogen for further 16 h. The mixture was filtered through celite and the solvent evaporated to give the hydrochloride salt of the title compound as a cream solid (102.5 g, 91%). ¹H-NMR (DMSO) δ 8.61 (3H, s), 7.93 (2H, d), 7.83 (2H, d), 7.80 (2H, d), 7.65 (2H, d), 4.08 (2H, s); MS(APCI+) found (M-NH₂)=235, C₁₄H₁₂F₃N requires 251.

### Intermediate A4—N-(1-(t-butoxycarbonyl)piperidin-4-yl)-4-(4-trifluoromethylphenyl)-benzylamine

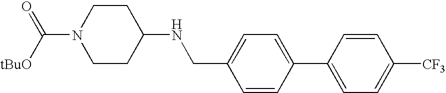

To a solution of intermediate A1 (11.2 g) and 4-amino-N-(t-butoxycarbonyl)piperidine (8.96 g) in dichloromethane was added dried 4 Å molecular sieves and the mixture stirred occaisionally over 18 h. The mixture was filtered and the solvent removed under reduced pressure. The residue was dissolved in ethanol and sodium triacetoxyborohydride (18.5 g) added. The mixture was stirred overnight at room temperature. The mixture was concentrated almost to dryness and partitioned between dichloromethane and water. The aqueous layer was extracted with further dichloromethane (×2) and the combined organic layers were evaporated under reduced pressure. The residue was chromatographed on silica gel using ethyl acetate as eluent. This gave the title compound as a white solid (10 g). ¹H-NMR (CDCl₃) δ 1.3-1.45 (2H, m), 1.45 (9H, s), 2.65-2.9 (3H, m), 3.5-3.8 (2H, br), 3.92 (2H, s), 3.95-4.2 (2H, br); 7.43 (2H, d), 7.57 (2H, d), 7.68 (4H, s).

Similarly prepared, except using sodium borohydride in place of sodium triacetoxyborohydride, was:

### Intermediate A5—N-(1-Benzylpiperidin-4-yl)-4′-trifluoromethylbiphen-4-ylmethylamine

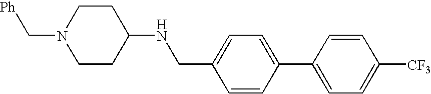

### Intermediate A6—8-Thiazol-2-ylmethyl-1,4-dioxa-8-aza-spiro[4.5]decane

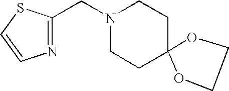

A mixture of 2-chloromethylthiazole hydrochloride (2.1 g), 1,4-dioxa-8-azaspiro[4.5]decane (1.58 ml) and diisopropylethylamine (4.73 ml) in dichloromethane (40 ml) was stirred at room temperature for 16 h at room temperature. The solution was washed with saturated sodium bicarbonate (×2) and dried over sodium sulphate. The solvent was removed under reduced pressure and the residue chromatographed on silica gel using 1-3% methanol in dichloromethane as eluents. This gave the desired product (1.75 g). ¹H-NMR (CDCl₃) δ 1.78 (4H, t) 2.68 (4H, t), 3.89 (2H, s), 3.95 (4H, s), 7.2-7.3 (1H, m), 7.65-7.75 (1H, m); MS(APCI+) found (M+1)=241, C₁₁H₁₆N₂O₂S requires 240.

Similarly prepared from 2-chloromethylpyridine and 1,4-dioxa-8-azaspiro[4.5]decane was:

### Intermediate A7—8-Pyrid-2-ylmethyl-1,4-dioxa-8-aza-spiro[4.5]decane

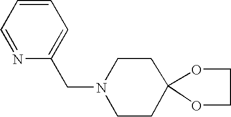

### Intermediate A8—1-Thiazol-2-ylmethylpiperidin-4-one

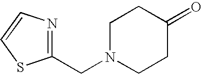

8-Thiazol-2-ylmethyl-1,4-dioxa-8-aza-spiro[4.5]decane (Int A6) (1.53 g) was dissolved in 2M hydrochloric acid (15 ml) and heated at 55° C. for 16 h. The mixture was cooled in ice and 40% sodium hydroxide was added to pH 12. The mixture was extracted with dichloromethane (×3), the combined extracts dried over sodium sulphate and evaporated under reduced pressure to give the desired product (1.26 g). ¹H-NMR (CDCl₃) δ 2.51 (4H, t) 2.91 (4H, t), 4.02 (2H, s), 7.34 (1H, d), 7.75 (1H, d).

Similarly prepared from intermediate A7 was:

### Intermediate A9—1-Pyrid-2-ylmethylpiperidin-4-one

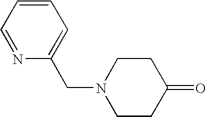

### Intermediate A10—1-Benzyloxycarbonylmethylpiperidin-4-one

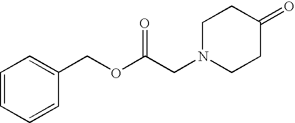

Piperidin-4-one hydrochloride (2.71 g) was suspended in dichloromethane and benzylbromoacetate (3.17 ml) and diisopropylethylamine (7.66 ml) added. The clear solution warmed slightly and was left for 5 h at room temperature. The mixture was diluted with dichloromethane and washed with saturated sodium bicarbonate and dried over sodium sulphate. Removal of the solvent gave the desired product (4.87 g). This material could be crystallised on trituration with light petrol. ¹H-NMR (CDCl₃) δ 2.50 (4H, t) 2.92 (4H, t), 3.42 (2H, s), 5.18 (2H, s), 7.25-7.45 (5H, m).

### Intermediate A11—N-(1-Thiazol-2-ylmethylpiperidin-4-yl)-4N-trifluoromethylbiphen-4-ylmethylamine

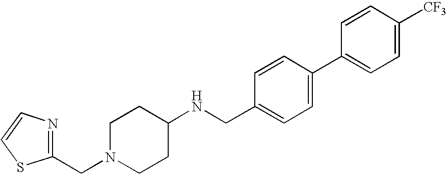

A mixture of 1-thiazol-2-ylmethylpiperidin-4-one (Int. A8) (1.23 g), 4-(4-trifluoromethylphenyl)-benzylamine hydrochloride salt (Int. A3), sodium triacetoxyborohydride (2.13 g) and acetic acid (0.376 ml) in dichloromethane was stirred under nitrogen for 40 h. The solution was poured into 2M sodium hydroxide (40 ml) with stirring. The organic layer was separated and the aqueous layer re-extracted with methylene chloride. The combined organic layers were dried over sodium sulphate and evaporated under reduced pressure. This gave a solid (2.57 g) that was chromatographed on silica gel using 24% methanol in dichloromethane as eluents. This gave the desired product (1.8 g). ¹H-NMR (CDCl₃) δ 1.85-2.0 (2H, m), 2.2-2.3 (2H, m), 2.5-2.65 (1H, m), 2.9-3.05 (2H, m), 3.87 (2H, s), 3.88 (2H s), 7.28 (1H, d), 7.43 (2H, d), 7.56 (2H, d), 7.6-7.8 (5H, m); LC/MS (LC conditions as for Example 1), (ESI+) found (M+1) 432, C₂₃H₂₄F₃N₃S requires 431. LC/MS purity=100%.

Similarly prepared from Int. A9 and Int. A3 was:

### Intermediate A12—N-(1-Pyrid-2-ylmethylpiperidin-4-yl)-4′-trifluoromethylbiphen-4-ylmethylamine

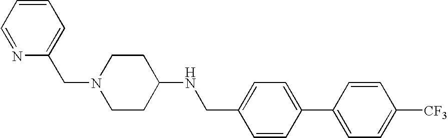

Similarly prepared from Int. A10 and A3, except using sodium bicarbonate in place of sodium hydroxide, was:

### Intermediate A13—N-(1-Benzyloxycarbonylmethylpiperdin-4-yl)-4′-trifluoromethylbiphen-4-ylmethylamine

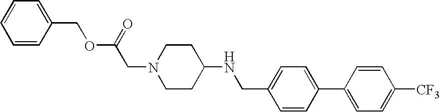

### Intermediate B1—4-Chloro-2-(2-(2,3-difluorophenyl)ethyl)quinoline

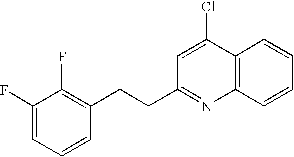

Butyllithium (4.76 ml, 2.5M in hexanes, 1 equiv) was added dropwise to a solution of 4-chloroquinaldine (2.4 ml, 1 equiv) in tetrahydrofuran (30 ml) at −78° C. and the reaction mixture stirred for 15 min. 2,3-Difluorobenzyl bromide (1.82 ml, 1.2 equiv) was added dropwise and stirring was continued for 1 h. After warming to room temperature the solution was diluted with water and ethyl acetate and the organic phase dried and evaporated. Chromatography (silica, 10:1 petrol/ethyl acetate) gave the title compound as a white solid (3.16 g). ¹H-NMR (CDCl₃) δ 3.23 (4H, m), 6.89-6.99 (3H, m), 7.33 (1H, s), 7.59 (1H, m), 7.74 (1H, m), 8.04 (1H, d), 8.15 (1H, d); MS (APCI+) found (M+1)=304; C₁₇H₁₂³⁵ClF₂N requires 303.

### Intermediate B2—2-(2-(2,3-Difluorophenyl)ethyl)-1H-quinolin-4-one

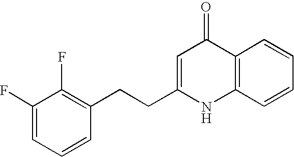

4-Chloro-2-(2,3-difluorophenylethyl)quinoline (Int. B1) (2.83 g) was heated to reflux in aqueous hydrochloric acid (2M, 15 ml) and dioxane (6 ml) for 72 h. The reaction mixture was extracted with dichloromethane (90 ml) and methanol (10 ml), and the organic phase dried and evaporated to give the title compound as a white solid (2.61 g). ¹H-NMR (d₆-DMSO) δ 3.15 (4H, s), 6.46 (1H, s), 7.15 (2H, m), 7.27 (1H, m), 7.51 (1H, m), 7.82 (2H, m), 8.15 (1H, d); MS (APCI+) found (M+1)=286; C₁₇H₁₃F₂NO requires 285.

### Intermediate B3—Tert butyl[2-(2-(2,3-difluorophenyl)ethyl)-4-oxo-4H-quinolin-1-yl]acetate

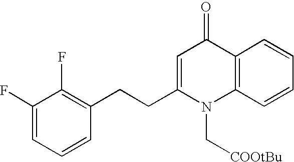

To a slurry of 2-(2-(2,3-Difluorophenyl)ethyl)-1H-quinolin-4-one (Int. B2) (33 g) in dry THF (500 ml) at 0° C. under argon was added dropwise n-butyl lithium (2.5M in hexanes) (47 ml). The solid dissolved during the addition. The mixture was allowed to warm to room temperature and stirred at this temperature for 0.5 h. Tert. butyl bromoacetate (28 ml) was added and the mixture heated at 40° C. for 48 h. The mixture was cooled to room temperature and poured into saturated ammonium chloride and extracted with dichloromethane (×3). The combined extracts were washed with brine, dried over magnesium sulphate and evaporated under reduced pressure to give a brown solid. Trituration of this material with hexane and then diethyl ether gave the title compound (35.7 g). ¹H NMR (CDCl₃) δ 1.46 (9H, s), 2.85-3.15 (4H, m), 4.83 (2H, s), 6.25 (1H, s), 6.8-7.2 (3H, m), 7.2-7.45 (2H, m), 7.6-7.7 (1H, m), 8.4-8.5 (1H, m).

### Intermediate B4—[2-(2-(2,3-Difluorophenyl)ethyl)-4-oxo-4H-quinolin-1-yl]acetic acid

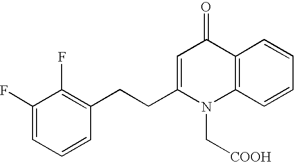

To a solution of tert butyl[2-(2-(2,3-difluorophenyl)ethyl)-4-oxo-4H-quinolin-1-yl]acetate (Int. B3) (35 g) in dry dichloromethane (300 ml) was added trifluoroacetic acid (50 ml) and the solution left for 68 h. The mixture was evaporated under reduced pressure to give an oily gum which was triturated with diethyl ether. The solid so formed was washed with water and dried under vacuum. This gave the desired material (30 g). ¹H-NMR (d₆-DMSO) δ 2.8-3.2 (4H, m), 5.24 (2H, s), 6.19 (1H, s), 7.05-7.4 (3H, m), 7.4-7.55 (1H, m), 7.55-7.9 (2H, m), 8.15-8.3 (1H, m)

### Intermediate C1—3-Azaisatoic anhydride

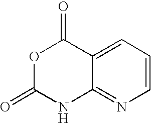

To a stirring solution of 2,3-pyridinedicarboxylic anhydride (10 g, 1 equiv) in anhydrous tetrahydrofuran (1 L) was added dropwise under argon at 38-46° C. over 1.25 h azidotrimethylsilane (97.9 ml, 1.1 equiv). The temperature was maintained at 45-50° C. for a further 2 h then the mixture refluxed for 30 min, cooled to ambient temperature and ethanol (43 ml, 1.1 equiv) added dropwise. On stirring for 16 h an off-white solid was obtained which was filtered, washed and dried, to give the title compound (90.7 g). ¹H-NMR (d₆-DMSO) δ 7.25-7.35 (1H, m), 8.30-8.35 (1H, dd), 8.65-8.7 (1H, dd), 11.3 (1H, br s).

### Intermediate C2—Ethyl(2,4-dioxo-4H-pyrido[2,3-d][1,3]oxazin-1-yl)acetate

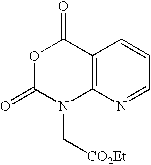

A 2:1 mixture of 3- and 6-azaisatoic anhydride (3.55 g, 21.6 mmol) (Synthesis 1982, 11, 972) was added portionwise to a suspension of sodium hydride (0.95 g, 60% in oil, 23.8 mmol) in DMF (40 ml). After stirring for 1 h, ethyl bromoacetate (2.64 ml, 23.8 mmol) was added. The reaction mixture was stirred overnight. The solvent was removed under reduced pressure. Ice/water was added to the residue and stirred for 1 h. The resulting pink solid was collected by filtration, washed with water and dried under vacuum at 40° C. The product was a 4:1 mixture of the [2,3-d] and the [3,2-d] isomers. ¹H-NMR data of the title compound. ¹H-NMR (d₆-DMSO)S 1.21 (3H, t), 4.18 (2H, q), 4.92 (2H, s), 7.45 (1H, dd), 8.47 (1H, dd), 8.77 (1H, dd); MS (APCI+) found (M+1)=251; C₁₁H₁₀N₂O₅ requires 250.

The title compound could also be prepared by the following method:

To a stirring mixture of 3-azaisatoic anhydride (Int. C1) (84.36 g, 1 equiv) and N,N-diisopropylethylamine (94 ml, 1.05 equiv) in N-methylpyrrolidone (420 ml) was added dropwise under argon at 45-50° C., ethyl bromoacetate (57 ml, 1 equiv). After 16 h at 50° C. the mixture was cooled (ice bath) and water (560 ml) added with vigorous stirring. The solid which precipitated was filtered, washed with water and partitioned between ethyl acetate and saturated aqueous sodium bicarbonate. An insoluble solid was filtered off and discarded and the ethyl acetate layer washed again with saturated sodium bicarbonate, dried (Na₂SO₄) and evaporated. The residue was triturated with a 1:1 mixture of ether/light petrol, filtered, washed and dried to give the title compound as an off-white solid, yield (56.0 g).

### Intermediate C₃₋₅-(2,3-difluorophenyl)-3-oxopentanoic acid tert-butyl ester

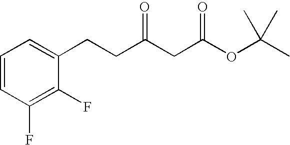

To an ice cooled stirring suspension of sodium hydride (1.96 g, 49.1 mmol, 60% dispersion in oil) in dry tetrahydrofuran (100 ml) was added dropwise under an argon atmosphere tert-butylacetoacetate (7.4 ml, 44.6 mmol). After a further 15 min, n-butyllithium (18.7 ml, 46.8 mmol, 2.5M in hexanes) was added dropwise maintaining the reaction temperature below 10° C. 2,3-Difluorobenzyl bromide (11.08 g, 53.5 mmol) was added dropwise 20 min later, then the mixture allowed to warm to ambient temperature. After a further 15 min the reaction mixture was poured onto a mixture of water (150 ml) and glacial acetic acid (10 ml), extracted 3 times with ethyl acetate and the combined extracts washed with saturated sodium hydrogen carbonate then brine, dried (MgSO₄) and evaporated to a yellow oil. Chromatography (fine silica, ethyl acetate-light petrol) gave the title compound as a yellow oil, yield 9.05 g (71%). ¹H NMR (CDCl₃) δ 1.45 (9H, s), 2.84-2.91 (2H, m), 2.95-3.00 (2H, m), 3.35 (2H, s), 6.92-7.04 (3H, m).

### Intermediate C4—(3-tert-butoxycarbonylmethyl-2-[2-(2,3-difluorophenyl)ethyl]-4-oxo-4H-[1,8]naphthyridin-1-yl)acetic acid ethyl ester

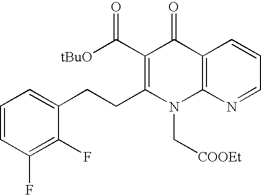

To a stirring suspension of sodium hydride (562 mg, 14.06 mmol, 60% dispersion in oil) in dry DMF (50 ml) was added dropwise 5-(2,3-difluorophenyl)-3-oxopentanoic acid tert-butyl ester (Int. C3) (3.63 g, 12.78 mmol). After 10 min, ethyl(2,4-dioxo-4H-pyrido[2,3-d][1,3]oxazin-1-yl)acetate (Int. C2) (3.21 g, 12.78 mmol) was added and the mixture stirred for 16 h. The solvent was evaporated and the residue treated with saturated aq. ammonium chloride and extracted 3 times with ethyl acetate. The combined extracts were washed with brine, dried (MgSO₄) and concentrated. Chromatography (fine silica, ethyl acetate-light petrol) gave the title compound as a light brown solid, yield 1.88 g (31%). ¹H NMR (d6-DMSO) δ 1.31 (3H, t), 1.63 (9H, s), 2.95-3.03 (2H, m), 3.08-3.13 (2H, m), 4.27 (2H, q), 5.31 (2H, s), 7.01-7.11 (3H, m), 7.35-7.38 (1H, m), 8.67-8.71 (2H, m).

The title compound was also made by the following method:

To an ice-cooled solution of intermediate C2 (55.9 g, 1 equiv) and intermediate C3 (63.5 g, 1 equiv) in dichloromethane (700 ml) was added dropwise under argon over 45 min 1,8-diazabicyclo[5.4.0]undec-7-ene (40 ml, 1.2 equiv). After 1 h the ice bath was removed and after a further 2.5 h the mixture was washed with saturated aqueous ammonium chloride, dried (Na₂SO₄) and evaporated. The crude product was chromatographed (fine silica, ethyl acetate-dichloromethane) then triturated with light petrol to give the title compound (80.27 g).

### Intermediate C5—(2-(2-(2,3-difluorophenyl)ethyl)-4-oxo-4H-[1,8]naphthyridin-1-yl)acetic acid ethyl ester

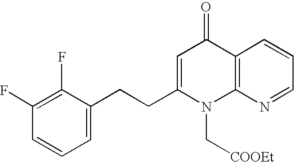

(3-tert-Butoxycarbonylmethyl-2-[2-(2,3-difluorophenyl)ethyl]4-oxo-4H-[1,8]naphthyridin-1-yl)acetic acid ethyl ester (Int C4) (1.35 g, 2.86 mmol) was added portionwise to boiling diphenyl ether (10 ml) with stirring. After 20 min, the dark solution was allowed to cool to ambient temperature. Petroleum ether (b.p. 60-80) was added to the point of cloudiness to give the product as a crystalline solid, yield 724 mg (68%). ¹H NMR (d6-DMSO) δ 1.19 (3H, t), 3.02-3.09 (4H, m), 4.16 (2H, q), 5.31 (2H, s), 6.10 (1H, s), 7.13-7.21 (2H, m), 7.26-7.33 (1H, m), 7.46-7.49 (1H, m), 8.49 (1H, m), 8.76 (1H, m). MS (APCI+), found (M+1)=373, C₂₀H₁₈F₂N₂O₃ requires 372.

The following intermediate was prepared by the method of Intermediate D4:

| No. | Precursor | Structure | Name |
|---|---|---|---|
| C6 | C5 | 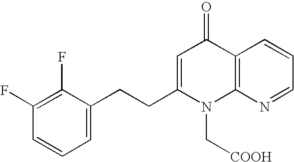 | (2-[2-(2,3-difluorophenyl)ethyl]-4-oxo-4H- [1,8]naphthyridin-1-yl)acetic acid |

### Intermediate D1—5-(1-(2,3-Difluorobenzylthio)-1-phenylaminomethylene)-2,2-dimethyl-[1,3]dioxane-4,6-dione

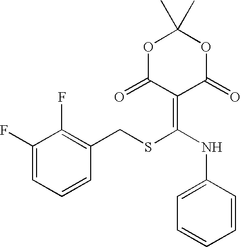

To hexane washed sodium hydride (7.45 g, 60% in oil) under argon, was added N-methylpyrrolidone (NMP) (270 ml) and the mixture cooled in an ice-salt bath. 2,2-Dimethyl-1,3-dioxane-4,6-dione (26.8 g) was added portionwise over 20 min keeping the temperature between 5-10° C. Effervescence was noted during the addition. The mixture was stirred at room temperature for 1 h and phenylisothiocyanate (25.2 g) added over 15 min. The mixture was stirred at room temperature for 2.5 h and cooled to 15° C. in a cold water bath. 2,3-Difluorobenzyl bromide (38.6 g) was added over 10 min and stirred at room temperature overnight. The solvent was removed under reduced pressure and the residue partioned between ethyl acetate (1.2 L) and water. The organic layer was washed with further water and then brine and dried over MgSO₄. The solvent was removed under reduced pressure and the residue triturated with 40-60° C. petrol and the solid collected by filtration. Crystallisation from methyl t.butyl ether gave the title compound as a pale yellow solid (51.4 g). ¹H-NMR (d₆-DMSO) δ 1.64 (6H, s), 4.16 (2H, d), 7.1-7.25 (2H, m), 7.25-7.5 (6H, m), 12.12 (1H, br s); MS (APCI−) found (M−1)=404; C₂₀H₁₇F₂NO₄S requires 405.

### Intermediate D2—Ethyl 2-(1-(2,3-difluorobenzylthio)-1-(2,2-dimethyl-4,6-dioxo-[1,3]dioxan-5-ylidene)-methyl)phenylamino)acetate

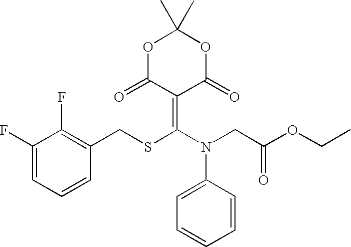

To hexane washed sodium hydride (1.0 g, 60% in oil) under argon, was added NMP (30 ml). A solution of 5-(1-(2,3-difluorobenzylthio)-1-phenylaminomethylene)-2,2-dimethyl-[1,3]dioxane-4,6-dione (10.0 g) (intermediate D1) in NMP (20 ml) was added by syringe over 15 min at room temperature and stirred for 30 min. Ethyl bromoacetate (4.5 g) was added and the mixture heated at 60° C. for 6 h. The mixture was partitioned between ethyl acetate and water and the aqueous layer extracted with further ethyl acetate. The combined organic layers were washed with further water and brine, dried over MgSO₄, and the solvent removed under reduced pressure. The orange oil so obtained was triturated with diethyl ether/40-60° C. petrol to give a solid that was collected by filtration. This solid was recrystallised from methyl t-butyl ether to give the title compound (7.37 g). ¹H-NMR (d₆-DMSO) δ 1.24 (3H, t), 1.55 (6H, br s), 4.19 (2H, q), 4.37 (2H, d), 4.81 (2H, br s), 6.85-7.5 (8H, 2xm).

### Intermediate D3—Ethyl(2-(2,3-difluorobenzylthio)-4-oxo-4H-quinolin-1-yl)acetate

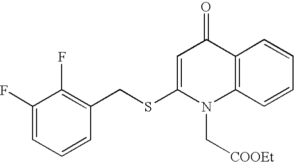

Ethyl(1-(2,3-difluorobenzylthio)-1-(2,2-dimethyl-4,6-dioxo-[1,3]dioxan-5-ylidene)methyl)phenylamino)acetate (intermediate D2) (0.85 g) under argon was stirred with trifluoroacetic (10 ml) at room temperature overnight. The mixture was evaporated under reduced pressure, dissolved in dichloromethane, washed with sodium bicarbonate solution and dried over Na₂SO₄. The solvent was removed under reduced pressure and the residue triturated with diethyl ether to give the title compound (0.43 g). ¹H-NMR (CDCl₃) δ 1.27 (3H, t), 4.26 (2H, q), 4.29 (2H, s), 5.1 (2H, br s), 6.45 (1H, s), 6.95-7.25 (4H, m), 7.39 (1H, t), 7.64 (1H, dt), 8.42 (1H, dd); MS (APCI+) found (M+1)=418; C₂₂H₂₁F₂NO₃S requires 417.

### Intermediate D4—[2-(2,3-Difluorobenzylthio)₄-oxo-4H-quinolin-1-yl]acetic acid

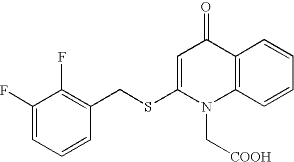

To a solution of Int. D3 (21.56 g, 0.055 mol) in dioxan (200 ml) was added sodium hydroxide (6.0 g, 0.15 mol) in water (200 ml) and the solution stirred for 2.5 h then concentrated. The residues were dissolved in water and acidified to pH 2 with 2M hydrochloric acid and the precipitate collected and washed sequentially with water, ether and then hexane. The solids were dried in vacuo at 40° C. to provide the title compound (20.0 g, 100%). ¹H-NMR (d₆-DMSO) δ 4.5 (2H, s), 5.2 (2H, br s), 6.3 (1H, s), 7.18 (1H, m), 7.3 (1H, m), 7.4 (2H, m), 7.6 (1H, d, J=8.5 Hz), 7.7 (1H, t, J=8 Hz), 8.1 (1H, d, J=8 Hz). MS (APCI+) found (M+1)=362; C₁₈H₁₃F₂NO₃S requires 361.

### Intermediate E1—3-(2,3-Difluorophenyl)propionic acid

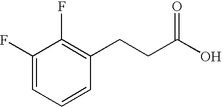

A solution of 2,3-difluorocinnamic acid (9.14 g) in ethanol (250 ml) with 10% palladium/carbon catalyst was hydrogenated for 5 h at room temperature and atmospheric pressure. The reaction mixture was filtered through celite and concentrated in vacuo to give the title compound as a colourless solid (9.05 g, quant.) ¹H-NMR (CDCl₃) δ 2.70 (2H, t, J=7.6 Hz), 3.02 (2H, t, J=7.6 Hz) and 7.01 (3H, m).

### Intermediate E2—Ethyl 2-(2-[2-(2,3-difluorophenyl)ethyl]-4-oxo-4H-quinazolin-1-yl)acetate

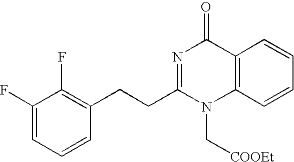

To a solution of 3-(2,3-difluorophenyl)propionic acid (Int. E1) (5 g, 26.88 mmol) in anhydrous dichloromethane (50 ml) containing a few drops of DMF was added oxalyl chloride (4.7 ml, 53.84 mmol) at 0° C. under argon. The solution was then stirred at ambient temperature for 2 h and the solvent removed in vacuo. The residue which contained the acid chloride was dissoved in toluene (50 ml) and added to a slurry of (2-carbamoylphenylamino)acetic acid ethyl ester (5.0 g, 22.52 mmol) in toluene (50 ml) containing pyridine (1 ml) and 4-dimethylaminopyridine (DMAP) (100 mg). After 16 h at 90° C. the solvent was evaporated and the solid residue washed with water, aqueous ammonia and ether to give the title compound (6.9 g, 82%) as a cream solid. ¹H-NMR (DMSO) δ 1.24 (3H, t), 3.13 (2H, t), 3.34 (2H, m), 4.24 (2H, q), 5.48 (2H, s), 7.19 (1H, m), 7.29-7.35 (2H, m), 7.60-7.72 (2H, m), 7.94 (1H, t), 8.19 (1H, d); MS (APCI+) found (M+1)=373; C₂₀H₁₈F₂N₂O₃ requires 372.

### Intermediate E3—2-(2-(2-(2,3-Difluorophenyl)ethyl)-4-oxo-4H-quinazolin-1-yl)acetic acid

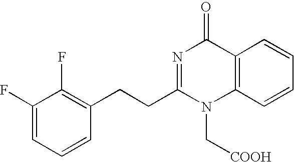

A solution of ethyl 2-(2-(2-(2,3-difluorophenyl)ethyl)-4-oxo-4H-quinazolin-1-yl)-acetate (Int. E2) (6.8 g, 18.3 mmol) in methanol (30 ml) and 2M sodium hydroxide solution (18.0 ml, 36 mmol) was stirred at ambient temperature overnight. The solvent was removed in vacuo and the residue dissolved in water (10 ml). Acidification to pH 1 with 2M hydrochloric acid gave a solid that was filtered, washed with water and dried in vacuo to give the desired product (5.9 g, 94%) as a white solid. ¹H-NMR (DMSO) δ 3.11-3.30 (4H, m), 5.31 (2H, s), 7.16-7.33 (3H, m), 7.61 (1H, t), 7.68 (1H, d), 7.89 (1H, t), 8.18 (1H, d); MS (APCI+) found (M+1)=345; C₁₈H₁₄F₂N₂O₃ requires 344.

### Intermediate F1—N-(1-(t-butoxycarbonyl)piperidin-4-yl)-2-[2-(2,3-difluorobenzylthio)-4-oxo-4H-quinolin-1-yl]-N-(4′-trifluoromethylbiphenyl-4-ylmethyl)acetamide

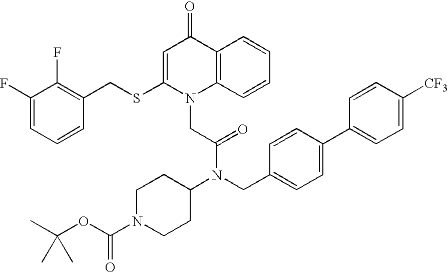

Prepared from Int. A4 and Int. D4 by the method of Example 1:

### Intermediate F2—N-(Piperidin-4-yl)-2-[2-(2,3-difluorobenzylthio)-4-oxo-4H-quinolin-1-yl]-N-(4′-trifluoromethylbiphenyl-4-ylmethyl)acetamide trifluoroacetate

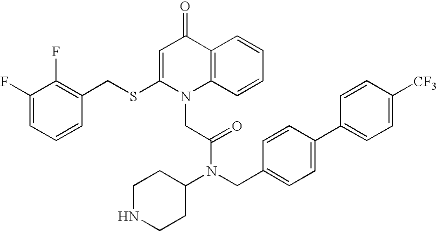

To intermediate F1 (6.4 g) in dichloromethane (20 ml) was added trifluoroacetic acid (14 ml) at room temperature. The mixture was stirred for 0.75 h, the solvent removed under reduced pressure and chromatographed on silica gel using 0-10% methanol:ethyl acetate. The crude product was dissolved in ethyl acetate and left to stand overnight. This gave the desired product as a solid (4.7 g). ¹H-NMR (d6 DMSO) δ 1.7-2.15 (4H, m), 2.85-3.1 (2H, m), 3.2-3.5 (2H, m), 4.25-5.9 (7H, br ms), 6.28+6.33 (1H, 2xs), 7.05-7.9 (14H, m), 8.15 (1H, ddd); LC/MS (ESI+) found (M+1)=678; C₃₄H₃₂F₅N₃O₂S requires 677. LC/MS purity=100%.

### Intermediate G1—2-[2-(4-Fluorobenzylthio)-5,6-trimethylene-4-oxo-4H-pyrimidin-1-yl]acetic acid

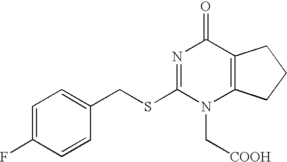

The preparation of this intermediate was described in International Application WO 01/60805 A1.

### Example 1

### N-(1-(Thiazol-2-ylmethyl)piperidin-4-yl)-2-[2-(2,3-difluorobenzylthio)-4-oxo-4N-quinolin-1-yl]-N-(4′-trifluoromethylbiphen-4-ylmethyl)acetamide

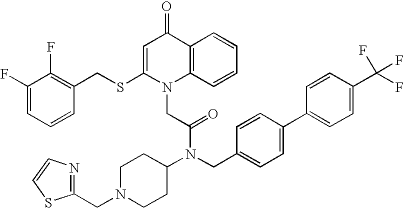

A mixture of 2-(2-(2,3-difluorobenzylthio)₄-oxo-4H-quinolin-1-yl)acetic acid (Int. D4) (0.837 g), N-(1-thiazol-2-ylmethylpiperidin-4-yl)-4′-trifluoromethylbiphen-4-ylmethylamine (Int. A11) (11.0 g), O-(7-azabenzotriazol-1-yl)-N,N,N′,N′-tetramethyluronium hexafluorophosphate (HATU) (1.23 g) and diisopropylamine (1.13 ml) in dimethylformamide (10 ml) was stirred at room temperature for 16 h. The solvent was removed under reduced pressure and the residue partioned between dichloromethane and 2M sodium hydroxide. The aqueous layer was extracted with further dichloromethane and the combined organic layers were washed with water, dried over anhydrous sodium sulphate and evaporated to a dark oil. This oil was chromatographed on silica gel using 24% 2M ammonia in methanol:ethyl acetate. Product fractions were evaporated and the residue triturated with ethyl acetate:diethyl ether to give the title compound (0.96 g). ¹H-NMR (CDCl₃) δ 1.7-2.15 (4H, m), 2.25-2.4 (2H, m), 2.95-3.2 (2H, m) 3.65-3.8+4.55-4.7 (1H, m) 3.85+3.92 (2H, 2xs), 4.22+4.27 (2H, 2xs), 4.71+4.76 (2H, 2xs), 4.9-5.55 (2H, m), 6.43+6.51 (1H, 2xs), 6.75-7.2 (4H, m), 7.2-7.4 (2H, m), 7.4-7.8 (9H, m) 8.3-8.5 (1H, m); LC/MS (LC conditions: 3.3 cm×4.6 mm ID, 3 μM ABZ+PLUS column using a gradient system 0.1% formic acid in 10 mM ammonium acetate:95% acetontrile with 0.05% formic acid, flow rate 3 ml/min, injection volume 5 μl), (ESI+) found (M+1) 775; C₄₁H₃₅F₅N₄O₂S₂ requires 774. LC/MS purity=100%.

### Example 2

### N-(1-(But-3-en-1-yl)piperidin-4-yl)-2-[2-(2,3-difluorobenzylthio)-4-oxo-4H-quinolin-1-yl]-N-(4′-trifluoromethylbiphen-4-ylmethyl)acetamide

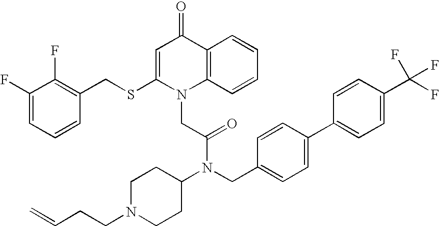

A mixture of N-(piperidin-4-yl)-2-[2-(2,3-difluorobenzylthio)₄-oxo-4H-quinolin-1-yl]-N-(4′-trifluoromethylbiphenyl-4-ylmethyl)acetamide trifluoroacetate (Int F2) (0.10 g), 4-chlorobut-1-ene (0.020 g), anhydrous potassium carbonate (0.081 g) and anhydrous sodium iodide (0.004 g) in dimethylformamide (2 ml) was heated at 100° C. The solvent was removed under reduced pressure and the residue partitioned between dichloromethane and water. The organic layer was separated, evaporated to dryness and purified using a Gilson Autoprep PLC on a 10 cm×2.1 cm SUPELCO ABZ+PLUS column containing SUPERCOSIL™ 5 M packing with a 0.1% trifluoroacetic acid in water:acetonitrile gradient system. The pure samples were partioned between saturated sodium bicarbonate and dichloromethane and the organic layer was evaporated to give the title compound (0.02 g). LC/MS (LC conditions as for Example 1), (ESI+) found (M+1) 732; C₄₁H₃₈F₅N₃O₂S requires 731. LC/MS purity=100%.

The following Examples were made by the general method of Example 1, using an appropriate solvent such as dimethylformamide or dichloromethane:

| Ex. | | | |
|---|---|---|---|
| No. | Precursors | Structure | Name |
| 3 | A13 B4 | 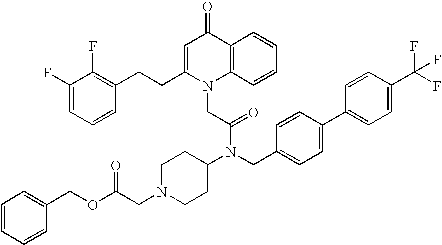 | N-(1-benzyloxycarbonylmethylpiperidin-4- yl)-2-[2-(2-(2,3-difluorophenyl)ethyl)-4- oxo-4H-quinolin-1-yl]-N-(4′-trifluoro- methylbiphen-4-ylmethyl)acetamide |
| 4 | A12 D4 | 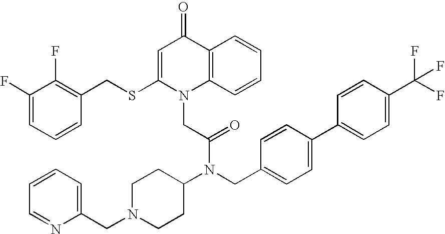 | N-(1-(pyrid-2-ylmethyl)piperidin-4-yl)-2- [2-(2,3-difluorobenzylthio)-4-oxo-4H- quinolin-1-yl]-N-(4′-trifluoromethylbiphen- 4-ylmethyl)acetamide bitartrate |
| 5 | A13 D4 | 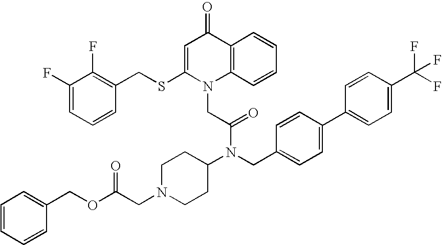 | N-(1-benzyloxycarbonylmethylpiperidin-4- yl)-2-[2-(2,3-difluorobenzylthio)-4-oxo-4H- quinolin-1-yl]-N-(4′-trifluoromethylbiphen- 4-ylmethyl)acetamide |
| 6 | A5 G1 | 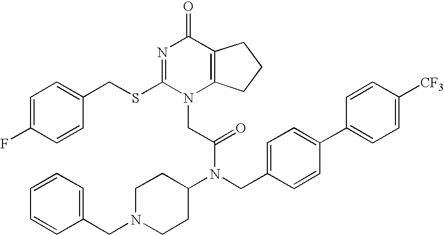 | N-(1-benzylpiperidin-4-yl)-2-[2-(4-fluoro- benzylthio)-4-oxo-5,6-trimethylene-4H- pyrimidin-1-yl]-N-(4′-trifluoromethyl- biphen-4-ylmethyl)acetamide bitartrate |
| 7 | A5 E3 | 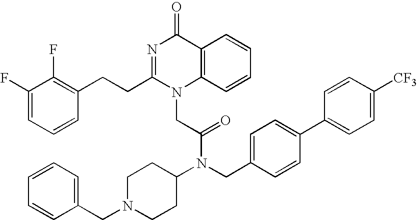 | N-(1-benzylpiperidin-4-yl)-2-[2-(2-(2,3- difluorophenyl)ethyl)-4-oxo-4H-quinazolin- 1-yl]-N-(4′-trifluoromethylbiphen-4-yl- methyl)acetamide |
| 8 | A5 C6 | 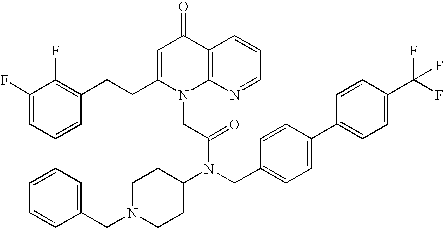 | N-(1-benzylpiperidin-4-yl)-2-[2-(2-(2,3- difluorophenyl)ethyl)-4-oxo-4H- [1,8]naphthyridin-1-yl]-N-(4′-trifluoro- methylbiphen-4-ylmethyl)acetamide |

The following Examples were made from Intermediate F2 and an appropriate alkylating agent by the general method of Example 2:

| Ex. | | |
|---|---|---|
| No. | Structure | Name |
| 10 | 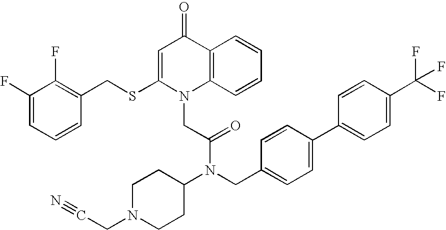 | N-(1-cyanomethylpiperidin-4-yl)-2-[2-(2,3-difluorobenzyl- thio)-4-oxo-4H-quinolin-1-yl]-N-4′-trifluoromethylbiphen- 4-ylmethyl)acetamide |
| 11 | 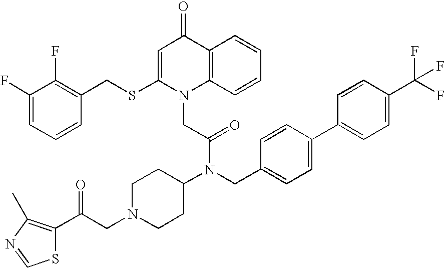 | N-(1-(4-methylthiazol-5-ylcarbonylmethyl)piperidin-4-yl)- 2-[2-(2,3-difluorobenzylthio)-4-oxo-4H-quinolin-1-yl]-N- (4′-trifluoromethylbiphen-4-ylmethyl)acetamide |
| 12 | 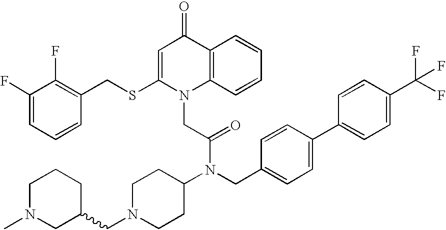 | (±) N-(1-(1-methylpiperid-3-ylmethyl)piperidin-4-yl)-2-[2- (2,3-difluorobenzylthio)-4-oxo-4H-quinolin-1-yl]-N-(4′- trifluoromethylbiphen-4-ylmethyl)acetamide |
| 13 | 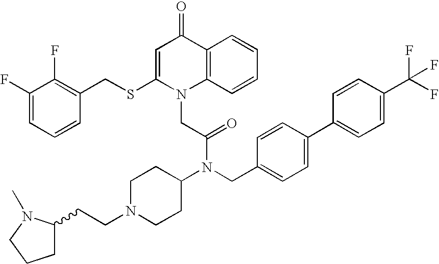 | (±) N-(1-(1-methylpyrrolid-2-ylethyl)piperidin-4-yl)-2-[2- (2,3-difluorobenzylthio)-4-oxo-4H-quinolin-1-yl]-N-(4′- trifluoromethylbiphen-4-ylmethyl)acetamide |
| 14 | 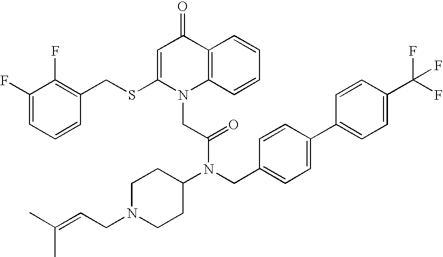 | N-(1-(3-methylbut-2-en-1-yl)piperidin-4-yl)-2-[2-(2,3- difluorobenzylthio)-4-oxo-4H-quinolin-1-yl]-N-(4′- trifluoromethylbiphen-4-ylmethyl)acetamide |
| 15 | 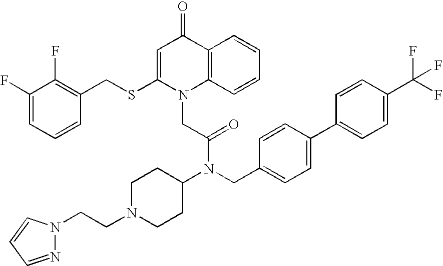 | N-(1-(2-(pyrazol-1-yl)ethyl)piperidin-4-yl)-2-[2-(2,3- difluorobenzylthio)-4-oxo-4H-quinolin-1-yl]-N-(4′- trifluoromethylbiphen-4-ylmethyl)acetamide |
| 16 | 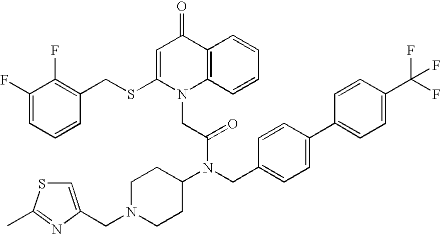 | N-(1-(2-methylthiazol-4-ylmethyl)piperidin-4-yl)-2-[2-(2,3- difluorobenzylthio)-4-oxo-4H-quinolin-1-yl]-N-(4′- trifluoromethylbiphen-4-ylmethyl)acetamide |
| 17 | 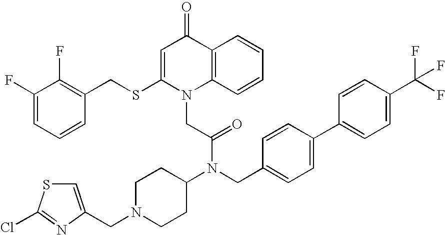 | N-(1-(2-chlorothiazol-4-ylmethyl)piperidin-4-yl)-2-[2-(2,3- difluorobenzylthio)-4-oxo-4H-quinolin-1-yl]-N-(4′- trifluoromethylbiphen-4-ylmethyl)acetamide |
| 18 | 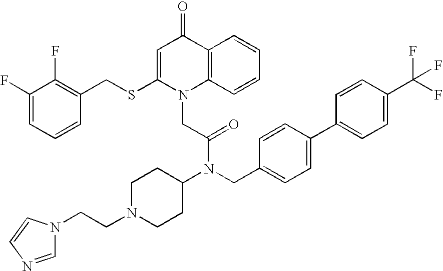 | N-(1-(2-(imidazol-1-yl)ethyl)piperidin-4-yl)-2-[2-(2,3- difluorobenzylthio)-4-oxo-4H-quinolin-1-yl]-N-(4′- trifluoromethylbiphen-4-ylmethyl)acetamide |
| 19 | 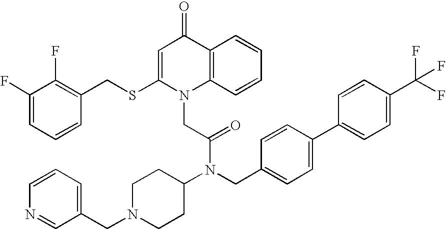 | N-(1-(pyrid-3-ylmethyl)piperidin-4-yl)-2-[2-(2,3-difluoro- benzylthio)-4-oxo-4H-quinolin-1-yl]-N-(4′-trifluoromethyl- biphen-4-ylmethyl)acetamide |
| 20 | 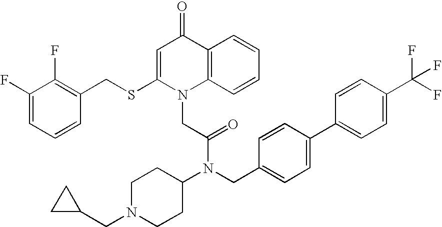 | N-(1-cyclopropylmethylpiperidin-4-yl)-2-[2-(2,3-difluoro- benzylthio)-4-oxo-4H-quinolin-1-yl]-N-(4′-trifluoromethyl- biphen-4-ylmethyl)acetamide |
| 21 | 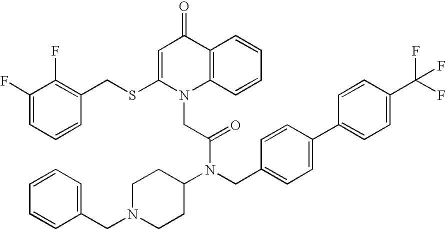 | N-(1-benzylpiperidin-4-yl)-2-[2-(2,3-difluorobenzylthio)-4- oxo-4H-quinolin-1-yl]-N-(4′-trifluoromethylbiphen-4-yl- methyl)acetamide |
| 22 | 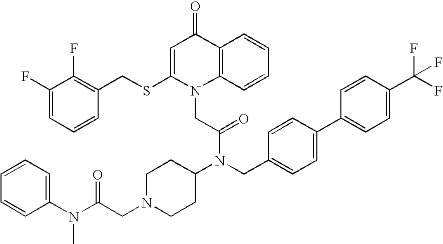 | N-(1-(N-phenyl-N-methylaminocarbonylmethyl)piperidin- 4-yl)-2-[2-(2,3-difluorobenzylthio)-4-oxo-4H-quinolin-1- yl]-N-(4′-trifluoromethylbiphen-4-ylmethyl)acetamide |
| 23 | 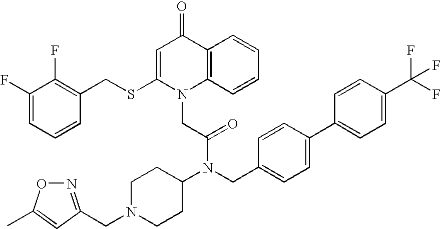 | N-(1-(5-methylisoxazol-3-ylmethyl)piperidin-4-yl)-2-[2- (2,3-difluorobenzylthio)-4-oxo-4H-quinolin-1-yl]-N-(4′- trifluoromethylbiphen-4-ylmethyl)acetamide |
| 24 | 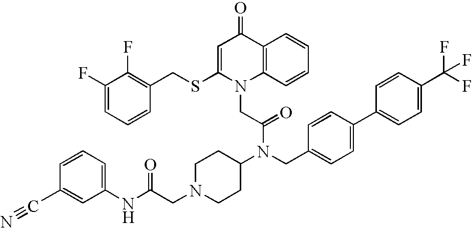 | N-(1-(N-(3-cyanophenyl)aminocarbonylmethyl)piperidin- 4-yl)-2-[2-(2,3-difluorobenzylthio)-4-oxo-4H-quinolin-1- yl]-N-(4′-trifluoromethylbiphen-4-ylmethyl)acetamide |
| 25 | 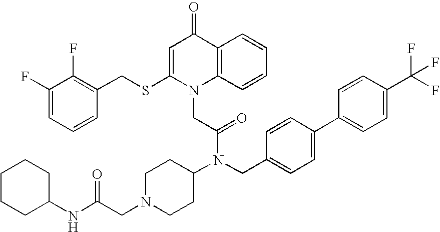 | N-(1-(cyclohexylaminocarbonylmethyl)piperidin-4-yl)-2- [2-(2,3-difluorobenzylthio)-4-oxo-4H-quinolin-1-yl]-N-(4′- trifluoromethylbiphen-4-ylmethyl)acetamide |
| 26 | 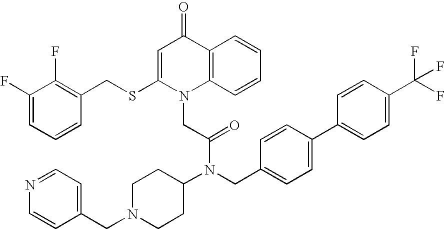 | N-(1-(pyrid-4-ylmethyl)pipendin-4-yl)-2-[2-(2,3-difluoro- benzylthio)-4-oxo-4H-quinolin-1-yl]-N-(4′-trifluoromethyl- biphen-4-ylmethyl)acetamide |
| 27 | 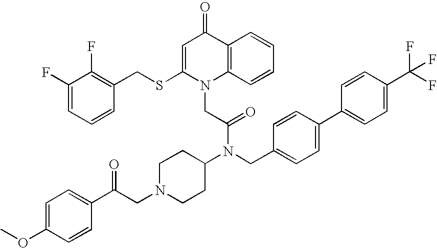 | N-(1-(N-(4-methoxyphenyl)carbonylmethyl)piperidin-4- yl)-2-[2-(2,3-difluorobenzylthio)-4-oxo-4H-quinolin-1-yl]- N-(4′-trifluoromethylbiphen-4-ylmethyl)acetamide |
| 28 | 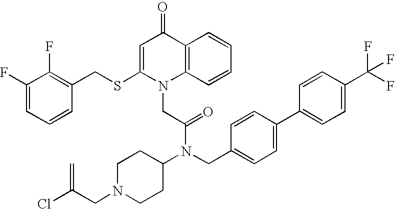 | N-(1-(2-chloroprop-2-en-1-yl)piperidin-4-yl)-2-[2-(2,3- difluorobenzylthio)-4-oxo-4H-quinolin-1-yl]-N-(4′- trifluoromethylbiphen-4-ylmethyl)acetamide |
| 29 | 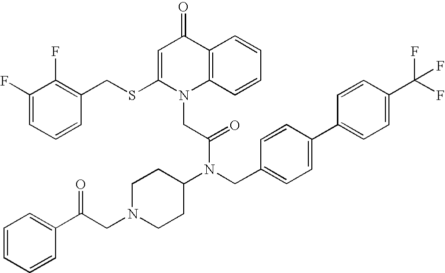 | N-(1-(2-oxo-2-phenyleth-1-yl)piperidin-4-yl)-2-[2-(2,3- difluorobenzylthio)-4-oxo-4H-quinolin-1-yl]-N-(4′- trifluoromethylbiphen-4-ylmethyl)acetamide |

Biological Data
1. Screen for Lp-PLA₂ Inhibition.

Enzyme activity was determined by measuring the rate of turnover of the artificial substrate (A) at 37 C in 50 mM HEPES (N-2-hydroxyethylpiperazine-N′-2-ethanesulphonic acid) buffer containing 150 mM NaCl, pH 7.4.

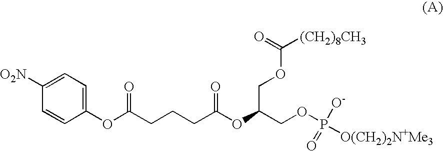

Assays were performed in 96 well titre plates.

Recombinant LpPLA2 was purified to homogeneity from baculovirus infected Sf9 cells, using a zinc chelating column, blue sepharose affinity chromatography and an anion exchange column. Following purification and ultrafiltration, the enzyme was stored at 6 mg/ml at 4° C. Assay plates of compound or vehicle plus buffer were set up using automated robotics to a volume of 1701 μl. The reaction was initiated by the addition of 20 μl of 10× substrate (A) to give a final substrate concentration of 20 μM and 10 μl of diluted enzyme to a final 0.1 nM LpPLA2.

The reaction was followed at 405 nm and 37° C. for 20 minutes using a plate reader with automatic mixing. The rate of reaction was measured as the rate of change of absorbance.

Results

The compounds described in the Examples were tested as described above and had IC₅₀ values in the range <0.1 to 100 nM.

1. A compound of formula (I): 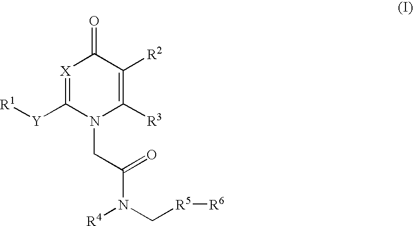
in which:
R¹ is an aryl group, optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR⁷COR⁸, CONR⁹R¹⁰, SO₂NR⁹R¹⁰, NR⁷SO₂R⁸, NR⁹R¹⁰, mono to perfluoro-C₍₁₋₄₎alkyl, mono to perfluoro-C₍₁₋₄₎alkoxyaryl, and arylC₍₁₋₄₎alkyl;
R² is halogen, C₍₁₋₃₎alkyl, C₍₁₋₃₎alkoxy, hydroxyC₍₁₋₃₎alkyl, C₍₁₋₃₎alkylthio, C₍₁₋₃₎alkylsulphinyl, aminoC₍₁₋₃₎alkyl, mono- or di-C₍₁₋₃₎alkylaminoC₍₁₋₃₎alkyl, C₍₁₋₃₎alkylcarbonylaminoC₍₁₋₃₎alkyl, C₍₁₋₃₎alkoxyC₍₁₋₃₎alkylcarbonylaminoC₍₁₋₃₎alkyl, C₍₁₋₃₎alkylsulphonylaminoC₍₁₋₃₎alkyl, C₍₁₋₃₎alkylcarboxy, C₍₁₋₃₎alkylcarboxyC₍₁₋₃₎alkyl, and
R³ is hydrogen, halogen, C₍₁₋₃₎alkyl, or hydroxyC₍₁₋₃₎alkyl; or
R² and R³ together with the pyridone or pyrimidone ring carbon atoms to which they are attached form a fused 5- or 6-membered carbocyclic ring; or
R² and R³ together with the pyridone or pyrimidone ring carbon atoms to which they are attached form a fused benzo or heteroaryl ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy or C₍₁₋₄₎alkylthio, or mono to perfluoro-C₍₁₋₄₎alkyl;
R⁴ is Het-C₍₀₋₄₎alkyl in which Het is a 5- to 7-membered saturated heterocyclyl ring comprising N and optionally O or S, and in which N is substituted by C₃₋₈cycloalkyl or C₍₁₋₆₎alkyl further substituted by 1, 2 or 3 substituents selected from R¹¹, COOR¹¹, COOCH₂R¹¹, COR¹¹, CN, CONR¹²R¹³, C₃₋₈cycloalkyl, vinyl optionally substituted by halogen or C₍₁₋₃₎alkyl and a 5- to 7-membered saturated heterocyclyl ring comprising N in which N may be substituted by C₁₋₃alkyl;
R⁵ is an aryl or a heteroaryl ring optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR⁷COR⁸, CONR⁹R¹⁰, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy;
R⁶ is an aryl or a heteroaryl ring which is further optionally substituted by 1, 2, 3 or 4 substituents which may be the same or different selected from C₍₁₋₆₎alkyl, C₍₁₋₆₎alkoxy, C₍₁₋₆₎alkylthio, C₍₁₋₆₎alkylsulfonyl, arylC₍₁₋₆₎alkoxy, hydroxy, halogen, CN, COR⁷, carboxy, COOR⁷, CONR⁹R¹⁰, NR⁷COR⁸, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy, or C₍₅₋₁₀₎alkyl;
R⁷ and R⁸ are independently hydrogen or C₍₁₋₁₂₎alkyl, for instance C₍₁₋₄₎alkyl (e.g. methyl or ethyl);
R⁹ and R¹⁰ which may be the same or different is each selected from hydrogen, or C₍₁₋₁₂₎alkyl, or R⁹ and R¹⁰ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl, e.g benzyl, for instance morpholine or piperazine;
R¹¹ is an unsubstituted 5- or 6-membered heteroaryl or an unsubstituted 6-membered aryl, or a 5- or 6-membered heteroaryl or a 6-membered aryl substituted by one or more R¹⁴.
R¹² is selected from hydrogen or C₁₋₃alkyl;
R¹³ is selected from phenyl optionally substituted by halogen, C₁₋₆alkyl, C₁₋₆alkoxy or cyano, or C₅₋₇cycloalkyl;
R¹⁴ is selected from the group consisting of halogen, CF₃, C₁₋₆alkyl, C₁₋₆alkoxy or cyano;
X is CH or nitrogen; and
Y is a C₍₂₋₄₎alkylene group (optionally substituted by 1, 2 or 3 substituents selected from methyl and ethyl), CH═CH, or (CH₂)ₙS where n is 1, 2 or 3, and a pharmaceutically acceptable salt thereof.

2. A compound according to claim 1 wherein R¹ is phenyl optionally substituted by 1, 2, 3 or 4 halogen substituents.

3. A compound according to claim 2 wherein R¹ is phenyl substituted by 1 to 3 fluoro.

4. A compound according to claim 1 wherein X is CH and R² and R³ together with the pyridone ring carbon atoms to which they are attached form a fused benzo or pyrido ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy or C₍₁₋₄₎alkylthio, or mono to perfluoro-C₍₁₋₄₎alkyl.

5. A compound according to claim 4 wherein the fused benzo or pyrido ring is unsubstituted.

6. A compound according to claim 1 wherein X is nitrogen and R² and R³ together with the pyrimidone ring carbon atoms to which they are attached form a fused 5-membered carbocyclic (cyclopentenyl) or benzo ring optionally substituted by 1, 2, 3 or 4 substituents which may be the same or different selected from halogen, C₍₁₋₄₎alkyl, cyano, C₍₁₋₃₎alkoxyC₍₁₋₃₎alkyl, C₍₁₋₄₎alkoxy, C₍₁₋₄₎alkylthio, or mono to perfluoro-C₍₁₋₄₎alkyl.

7. A compound according to claim 6 wherein the fused 5-membered carbocyclic or benzo ring is unsubstituted.

8. A compound according to claim 1 wherein R⁴ is Het C₍₀₎alkyl in which Het is a six-membered saturated heterocyclyl ring comprising nitrogen in which the nitrogen is substituted by C₃₋₈cycloalkyl or C₍₁₋₂₎alkyl substituted by a single substituent selected from R¹¹, COOR¹¹, COOCH₂R¹¹, COR¹¹, CN, CONR¹²R¹³, C₃₋₈cycloalkyl, vinyl optionally substituted by halogen or methyl and a 5- or 6-membered saturated heterocyclyl ring comprising N in which the nitrogen may be substituted by methyl.

9. A compound according to claim 1 wherein R⁵ is phenyl and R⁶ is phenyl substituted by mono to perfluoro-C₍₁₋₄₎alkyl, halogen or C₍₁₋₆₎alkyl.

10. A compound according to claim 9 wherein R⁶ is phenyl substituted by trifluoromethyl.

11. A compound to claim 1 wherein Y is CH₂S.

12. (canceled)

13. A pharmaceutical composition comprising a compound of formula (I) according to claim 1 and a pharmaceutically acceptable carrier, optionally with one or more other therapeutic compounds.

14. (canceled)

15. (canceled)

16. A method of treating a disease associated with activity of the enzyme Lp-PLA₂ which method involves treating a patient in need thereof with a therapeutically effective amount of a compound of formula (I) according to claim 1.

17. A process for preparing a compound of formula (I) which process comprises reacting an acid compound of formula (II): 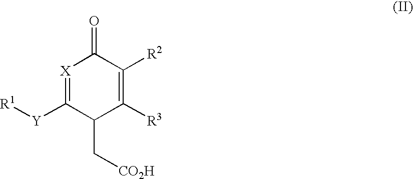
in which X, Y, R¹, R² and R³ are as hereinbefore defined,
with an amine compound of formula (III):
R⁶—R⁵—CH₂NHR⁴ (III)
in which R⁴, R⁵ and R⁶ are as hereinbefore defined; under amide forming conditions.
